(19) United States
(12) Patent Application Publication
(10) Pub. No.: US 2005/0112135 A1
(43) Pub. Date: May 26, 2005

(54) Ultrasound assisted transdermal vaccine delivery method and system

(76) Inventors: Michel J.N. Cormier, Mountain View, CA (US); WeiQi Lin, Palo Alto, CA (US); Georg Widera, Palo Alto, CA (US)

Correspondence Address: Francis Law Group, 1942 Embarcadero, Oakland, CA, 94606, US

(21) Appl. No.: 10/971,338

(22) Filed: Oct. 21, 2004

Related U.S. Application Data
(60) Provisional application No. 60/524,062, filed on Nov. 21, 2003.

(51) Int. Cl. A61K 39/12; A61M 31/00

(52) U.S. Cl. 424/185.1; 604/500

(57) ABSTRACT

An apparatus and method for transdermally delivering a vaccine comprising a delivery system having (i) a microprojection member (or system) that includes a plurality of microprojections (or array thereof) that are adapted to pierce through the stratum corneum into the underlying epidermis layer, or epidermis and dermis layers and (ii) an ultrasonic device. In one embodiment, the vaccine is contained in a biocompatible coating that is applied to the microprojection member. In a further embodiment, the delivery system includes a gel pack having a vaccine-containing hydrogel formulation that is disposed on the microprojection member after application to the skin of a patient. In an alternative embodiment, the vaccine is contained in both the coating and the hydrogel formulation.

### CROSS-REFERNCE TO RELATED APPLICATIONS

This application claims the benefit of U.S. Provisional Application No. 60/524,062, filed Nov. 21, 2003.

### FIELD OF THE PRESENT INVENTION

The present invention relates generally to transdermal vaccine delivery systems and methods. More particularly, the invention relates to an ultrasound assisted vaccine delivery method and system

### BACKGROUND OF THE INVENTION

Active agents (or drugs) are most conventionally administered either orally or by injection. Unfortunately, many active agents are completely ineffective or have radically reduced efficacy when orally administered, since they either are not absorbed or are adversely affected before entering the bloodstream and thus do not possess the desired activity. On the other hand, the direct injection of an agent into the bloodstream, while assuring no modification of the agent during administration, is a difficult, inconvenient, painful and uncomfortable procedure which sometimes results in poor patient compliance.

Hence, in principle, transdermal delivery provides for a method of administering active agents that would otherwise need to be administered orally, by hypodermic injection or by intravenous infusion. Transdermal delivery, when compared to oral delivery, avoids the harsh environment of the digestive tract, bypasses gastrointestinal drug metabolism, reduces first-pass effects, and avoids the possible deactivation by digestive and liver enzymes.

The word “transdermal”, as used herein, is generic term that refers to delivery of an active agent (e.g., a therapeutic agent, such as a drug or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources, such as electricity (e.g., iontophoresis) and ultrasound (e.g., phonophoresis).

As is well known in the art, skin is not only a physical barrier that shields the body from external hazards, but is also an integral part of the immune system. The immune function of the skin arises from a collection of residential cellular and humoral constituents of the viable epidermis and dermis with both innate and acquired immune functions, collectively known as the skin immune system.

One of the most important components of the skin immune system are the Langerhan's cells (LC), which are specialized antigen presenting cells found in the viable epidermis. LC's form a semi-continuous network in the viable epidermis due to the extensive branching of their dendrites between the surrounding cells. The normal function of the LC's is to detect, capture and present antigens to evoke an immune response to invading pathogens. LC's perform his function by internalizing epicutaneous antigens, trafficking to regional skin-draining lymph nodes, and presenting processed antigens to T cells.

The effectiveness of the skin immune system is responsible for the success and safety of vaccination strategies that have been targeted to the skin. Vaccination with a live-attenuated smallpox vaccine by skin scarification has successfully led to global eradication of the deadly small pox disease. Intradermal injection using ⅕ to 1/10 of the standard IM doses of various vaccines has been effective in inducing immune responses with a number of vaccines while a low-dose rabies vaccine has been commercially licensed for intradermal application.

Transdermal delivery offers significant advantages for vaccination, given the function of the skin as an immune organ. Pathogens entering the skin are confronted with a highly organized and diverse population of specialized cells capable of eliminating microorganisms through a variety of mechanisms. Epidermal Langerhans cells are potent antigen-presenting cells. Lymphocytes and dermal macrophages percolate throughout the dermis. Keratinocytes and Langerhans cells express or can be induced to generate a diverse array of immunologically active compounds. Collectively, these cells orchestrate a complex series of events that ultimately control both innate and specific immune responses.

It is further thought that non-replicating antigens (i.e., killed viruses, bacteria, an subunit vaccines) enter the endosomal pathway of antigen presenting cells. The antigens are processed and expressed on the cell surface in association with class II MHC molecules, leading to the activation of CD4⁺ T cells. Experimental evidence indicates that introduction of antigens exogenously induces little or no cell surface antigen expression associated with class I MHC, resulting in ineffective CD8⁺ T activation. Replicating vaccines, on the other hand (e.g., live, attenuated viruses, such as polio and smallpox vaccines) lead to effective humoral and cellular immune responses and are considered the “gold standard” among vaccines. A similar broad immune response spectrum can be achieved by DNA vaccines.

In contrast, polypeptide based vaccines, like subunit vaccines, and killed viral and bacterial vaccines do elicit predominantly a humoral response, as the original antigen presentation occurs via the class II MHC pathway. A method to enable the presentation of these vaccines also via the class I MHC pathway would be of great value, as it would widen the immune response spectrum.

Several reports have suggested that soluble protein antigens can be formulated with surfactants, leading to antigen presentation via the class I pathway and induce antigen-specific class I-restricted CTLs (Raychaudhuri, et al., 1992). Introduction of protein antigen by osmotic lysis of pinosomes has also been demonstrated to lead to a class I antigen-processing pathway (Moore, et al.). Ultrasound techniques have been used to introduce macromolecules into cells in vitro and in vivo, and, particularly, DNA-based therapeutics. Studies with plasmid DNA have clearly demonstrated that the delivery efficiency can be significantly enhanced when ultrasound is employed.

There is, however, no published literature regarding in vivo intracellular ultrasound delivery of protein-based vaccines into skin antigen-presenting cells (APC) that leads to cellular loading of the protein onto class I MHC/HLA presentation molecules in addition to class II MHC/HLA presentation molecules. In particular, there is no mention of the use of a microprojection array in conjunction with ultrasound to achieve this means.

There is also no published literature mentioning the use of a microprojection array in conjunction with ultrasound to achieve in vivo delivery of a DNA vaccine intracellularly and subsequent cellular expression and loading of the protein onto class I MHC/HLA presentation molecules in addition to class II MHC/HLA presentation molecules.

As is well known in the art, the transdermal drug flux is dependent upon the condition of the skin, the size and physical/chemical properties of the drug molecule, and the concentration gradient across the skin. Because of the low permeability of the skin to many drugs, transdermal delivery has had limited applications. This low permeability is attributed primarily to the stratum corneum, the outermost skin layer which consists of flat, dead cells filled with keratin fibers (keratinocytes) surrounded by lipid bilayers. This highly-ordered structure of the lipid bilayers confers a relatively impermeable character to the stratum corneum.

One common method of increasing the passive transdermal diffusional agent flux involves pre-treating the skin with, or co-delivering with the agent, a skin permeation enhancer. A permeation enhancer, when applied to a body surface through which the agent is delivered, enhances the flux of the agent therethrough. However, the efficacy of these methods in enhancing transdermal protein flux has been limited, particularly for the larger proteins due to their size.

There also have been many techniques and systems developed to mechanically penetrate or disrupt the outermost skin layers thereby creating pathways into the skin in order to enhance the amount of agent being transdermally delivered. Illustrative are skin scarification devices, or scarifiers, which typically provide a plurality of tines or needles that are applied to the skin to scratch or make small cuts in the area of application. The vaccine is applied either topically on the skin, such as disclosed in U.S. Pat. No. 5,487,726, or as a wetted liquid applied to the scarifier tines, such as disclosed in U.S. Pat. Nos. 4,453,926, 4,109,655, and 3,136,314.

A major drawback associated with the use of a scarifier to deliver an active agent, such as a vaccine, is the difficulty in determining the transdermal agent flux and the resulting dosage delivered. Also, due to the elastic, deforming and resilient nature of skin to deflect and resist puncturing, the tiny piercing elements often do not uniformly penetrate the skin and/or are wiped free of a liquid coating of an agent upon skin penetration.

Other systems and apparatus that employ tiny skin piercing elements to enhance transdermal drug delivery are disclosed in U.S. Pat. Nos. 5,879,326, 3,814,097, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

The disclosed systems further typically include a reservoir for holding the agent and also a delivery system to transfer the agent from the reservoir through the stratum corneum, such as by hollow tines of the device itself. One example of such a device is disclosed in WO 93/17754, which has a liquid agent reservoir. The reservoir must, however, be pressurized to force the liquid agent through the tiny tubular elements and into the skin. Disadvantages of such devices include the added complication and expense for adding a pressurizable liquid reservoir and complications due to the presence of a pressure-driven delivery system.

As disclosed in U.S. patent application Ser. No. 10/045,842, which is fully incorporated by reference herein, it is also possible to have the active agent that is to be delivered coated on the microprojections instead of contained in a physical reservoir. This eliminates the necessity of a separate physical reservoir and developing an agent formulation or composition specifically for the reservoir.

A drawback of the coated microprojection systems is that they are generally limited to delivery of a few hundred micrograms of the agent. A further drawback is that they are limited to a bolus-type agent delivery profile.

Active transport systems have also been employed to enhance agent flux through the stratum corneum. One such system for transdermal agent delivery is referred to as “electrotransport”. The noted system employs an electric potential, which results in the application of electric current is aid in the transport of the agent through the stratum corneum.

A further active transport system, commonly referred to as “phonophoresis”, employs ultrasound (i.e., sound waves) to aid in the transport of the agent through the stratum corneum. Illustrative are the systems disclosed in U.S. Pat. No. 5,733,572 and patent Pub. No. 2002/0099356 A1.

In U.S. Pat. No. 5,733,572, an active system is disclosed that includes gas-filled microspheres as topical and subcutaneous delivery vehicles. The microspheres are made to encapsulate agents and are injected or otherwise administered to a patient. Ultrasonic energy is then used to rupture the microspheres to release the agent.

The ultrasound applied to the microspheres has a frequency in the range of 0.5 MHz and 10 MHz. This range of frequencies has, however, been shown to be of limited use in producing cavitation effects in skin cells, which are much larger than the size of typical microspheres.

In patent Pub. No. 2002/0099356, a further active system is disclosed. The noted system includes a “microneedle array” that utilizes sonic energy to deliver or extract biomolecules through membranes. The noted reference does not, however, teach or suggest the delivery of a vaccine. In particular, there is no description of a preparation that contains an infectious agent or its components, or a nucleic acid coding for these components, which is administered to stimulate an immune response that will protect or treat a person from illness due to that agent.

The '356 reference further does not teach or suggest the delivery of a vaccine or any other biologically active agent via coated microprojections.

It would therefore be desirable to provide an ultrasound assisted vaccine delivery system that employs microprojections and arrays thereof having a biocompatible coating that includes the vaccine that is to be delivered.

It is therefore an object of the present invention to provide a vaccine delivery method and system that substantially reduces or eliminates the aforementioned drawbacks and disadvantages associated with prior art agent delivery systems.

It is another object of the present invention to provide a vaccine delivery method and system that includes microprojections coated with a biocompatible coating that includes a vaccine.

It is yet another object of the present invention to provide an ultrasound vaccine delivery method and system that increases cellular uptake of DNA and polypeptide-based vaccine.

### SUMMARY OF THE INVENTION

In accordance with the above objects and those that will be mentioned and will become apparent below, the delivery system for transdermally delivering an immunologically active agent to a subject comprises a microprojection member having a plurality of stratum corneum-piercing microprojections, a formulation having the immunologically active agent; and an ultrasonic device adapted to apply ultrasonic energy to said subject.

In one embodiment of the invention, the microprojection member has a microprojection density of at least approximately 10 microprojections/cm², more preferably, in the range of at least approximately 200-2000 microprojections/cm².

In one embodiment of the invention, the microprojection member has microprojections adapted to pierce through the stratum corneum to a depth of less than about 500 micrometers.

In one embodiment, the microprojection member is constructed out of stainless steel, titanium, nickel titanium alloys, or similar biocompatible materials.

In an alternative embodiment, the microprojection member is constructed out of a non-conductive material, such as a polymer. Alternatively, the microprojection member can be coated with a non-conductive material, such as parylene.

Suitable immunologically active agents, antigenic agents or vaccines, can include viruses and bacteria, protein-based vaccines, polysaccharide-based vaccine, and nucleic acid-based vaccines.

Antigenic agents include, without limitation, antigens in the form of proteins, polysaccharide conjugates, oligosaccharides, and lipoproteins. These subunit vaccines include Bordetella pertussis (recombinant DPT vaccine—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitis, pseudomonas aeruginosa, streptococcus pneumoniae, treponema pallidum, and vibrio cholerae, and mixtures thereof.

Additional commercially available vaccines, which contain antigenic agents, include, without limitation, flu vaccines, Lyme disease vaccine, rabies vaccine, measles vaccine, mumps vaccine, chicken pox vaccine, small pox vaccine, hepatitis vaccine, pertussis vaccine, and diptheria vaccine.

Vaccines comprising nucleic acids include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct. The antigen that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include aluminum phosphate gel; aluminum hydroxide; algal glucan: β-glucan; cholera toxin B subunit; CRL1005: ABA block polymer with mean values of x=8 and y=205; gamma insulin: linear (unbranched) β-D(2->1) polyfructofuranoxyl-α-D-glucose; Gerbu adjuvant: N-acetylglucosamine-(β 1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂O (MTP); Murametide: Nac-Mur-L-Ala-D-Gln-OCH₃; Pleuran: β-glucan; QS-21; S-28463: 4-amino-a, a-dimethyl-1H-imidazo[4,5-c]quinoline-1-ethanol; sclavo peptide: VQGEESNDK.HCl (IL-1β 163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

In one embodiment of the invention, the microprojection member includes a biocompatible coating that is disposed on at least the microprojections.

The coating formulations applied to the microprojection member to form solid coatings can comprise aqueous and non-aqueous formulations having at least one immunologically active agent, which can be dissolved within a biocompatible carrier or suspended within the carrier.

In one embodiment of the invention, the coating formulations include at least one surfactant, which can be zwitterionic, amphoteric, cationic, anionic, or nonionic. Examples of suitable surfactants include sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives, such as sorbitan laureate, and alkoxylated alcohols such as laureth-4.

In one embodiment of the invention, the concentration of the surfactant is in the range of approximately 0.001-2 wt. % of the coating solution formulation.

In a further embodiment of the invention, the coating formulations include at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties is preferably in the range of approximately 0.01-20 wt. %, more preferably, in the range of approximately 0.03-10 wt. % of the coating.

In another embodiment, the coating formulations include a hydrophilic polymer selected from the following group: poly(vinyl alcohol), poly(ethylene oxide), poly(2-hydroxyethylmethacrylate), poly(n-vinyl pyrolidone), polyethylene glycol and mixtures thereof, and like polymers.

In a preferred embodiment, the concentration of the hydrophilic polymer in the coating formulation is in the range of approximately 0.01-20 wt. %, more preferably, in the range of approximately 0.03-10 wt. % of the coating formulation.

In another embodiment of the invention, the coating formulations include a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose and stachyose.

Preferably, the concentration of the biocompatible carrier in the coating formulation is in the range of approximately 2-70 wt. %, more preferably, in the range of approximately 5-50 wt. % of the coating formulation.

In a further embodiment, the coating formulations include a stabilizing agent, which can comprise, without limitation, a non-reducing sugar, a polysaccharide, a reducing or a DNase inhibitor.

In another embodiment, the coating formulations include a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating.

In yet another embodiment of the invention, the coating formulations include at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In a further embodiment of the invention, the coating formulation includes at least one antioxidant, which can be sequestering such as sodium citrate, citric acid, EDTA (ethylene-dinitrilo-tetraacetic acid) or free radical scavengers such as ascorbic acid, methionine, sodium ascorbate, and the like. Presently preferred antioxidants include EDTA and methionine.

In certain embodiments of the invention, the viscosity of the coating formulation is enhanced by adding low volatility counterions. In one embodiment, the agent has a positive charge at the formulation pH and the viscosity-enhancing counterion comprises an acid having at least two acidic pKas. Suitable acids include maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, aspartic acid, glutamic acid, carbonic acid, sulfuric acid, and phosphoric acid.

Another preferred embodiment is directed to a viscosity-enhancing mixture of counterions wherein the agent has a positive charge at the formulation pH and at least one of the counterion is an acid having at least two acidic pKas. The other counterion is an acid with one or more pKas. Examples of suitable acids include hydrochloric acid, hydrobromic acid, nitric acid, sulfuric acid, maleic acid, phosphoric acid, benzene sulfonic acid, methane sulfonic acid, citric acid, succinic acid, glycolic acid, gluconic acid, glucuronic acid, lactic acid, malic acid, pyruvic acid, tartaric acid, tartronic acid, fumaric acid, acetic acid, propionic acid, pentanoic acid, carbonic acid, malonic acid, adipic acid, citraconic acid, levulinic acid, glutaric acid, itaconic acid, meglutol, mesaconic acid, citramalic acid, citric acid, aspartic acid, glutamic acid, tricarballylic acid and ethylenediaminetetraacetic acid.

Generally, in the noted embodiments of the invention, the amount of counterion should neutralize the charge of the antigenic agent. In such embodiments, the counterion or the mixture of counterion is present in amounts necessary to neutralize the charge present on the agent at the pH of the formulation. Excess of counterion (as the free acid or as a salt) can be added to the formulation in order to control pH and to provide adequate buffering capacity.

In another preferred embodiment, the agent has a positive charge and the counterion is a viscosity-enhancing mixture of counterions chosen from the group of citric acid, tartaric acid, malic acid, hydrochloric acid, glycolic acid, and acetic acid. Preferably, counterions are added to the formulation to achieve a viscosity in the range of about 20-200 cp.

In a preferred embodiment, the viscosity-enhancing counterion is an acidic counterion such as a low volatility weak acid. Low volatility weak acid counterions present at least one acidic pKa and a melting point higher than about 50° C. or a boiling point higher than about 170° C. at Pₐₜₘ. Examples of such acids include citric acid, succinic acid, glycolic acid, gluconic acid, glucuronic acid, lactic acid, malic acid, pyruvic acid, tartaric acid, tartronic acid, and fumaric acid.

In another preferred embodiment the counterion is a strong acid. Strong acids can be defined as presenting at least one pKa lower than about 2. Examples of such acids include hydrochloric acid, hydrobromic acid, nitric acid, sulfonic acid, sulfuric acid, maleic acid, phosphoric acid, benzene sulfonic acid and methane sulfonic acid.

Another preferred embodiment is directed to a mixture of counterions wherein at least one of the counterion is a strong acid and at least one of the counterion is a low volatility weak acid.

Another preferred embodiment is directed to a mixture of counterions wherein at least one of the counterions is a strong acid and at least one of the counterion is a weak acid with high volatility. Volatile weak acid counterions present at least one pKa higher than about 2 and a melting point lower than about 50° C. or a boiling point lower than about 170° C. at Pₐₜₘ. Examples of such acids include acetic acid, propionic acid, pentanoic acid and the like.

Preferably, the acidic counterion is present in amounts necessary to neutralize the positive charge present on the antigenic agent at the pH of the formulation. Excess of counterion (as the free acid or as a salt) can be added to the formulation in order to control pH and to provide adequate buffering capacity.

In yet other embodiments of the invention, particularly where the antigenic agent has a negative charge, the coating formulation further comprises a low volatility basic counter ion.

In a preferred embodiment, the coating formulation comprises a low volatility weak base counterion. Low volatility weak bases present at least one basic pKa and a melting point higher than about 50° C. or a boiling point higher than about 170° C. at Pₐₜₘ. Examples of such bases include monoethanolomine, diethanolamine, triethanolamine, tromethamine, methylglucamine, and glucosamine.

In another embodiment, the low volatility counterion comprises a basic zwitterions presenting at least one acidic pKa, and at least two basic pKa's, wherein the number of basic pKa's is greater than the number of acidic pkA's. Examples of such compounds include histidine, lysine, and arginine.

In yet other embodiments, the low volatility counterion comprises a strong base presenting at least one pKa higher than about 12. Examples of such bases include sodium hydroxide, potassium hydroxide, calcium hydroxide, and magnesium hydroxide.

Other preferred embodiments comprise a mixture of basic counterions comprising a strong base and a weak base with low volatility. Alternatively, suitable counterions include a strong base and a weak base with high volatility. High volatility bases present at least one basic pKa lower than about 12 and a melting point lower than about 50° C. or a boiling point lower than about 170° C. at Pₐₜₘ. Examples of such bases include ammonia and morpholine.

Preferably, the basic counterion is present in amounts necessary to neutralize the negative charge present on the antigenic agent at the pH of the formulation. Excess of counterion (as the free base or as a salt) can be added to the formulation in order to control pH and to provide adequate buffering capacity.

Preferably, the coating formulations have a viscosity less than approximately 500 centipoise and greater than 3 centipoise.

In one embodiment of the invention, the coating thickness is less than 25 microns, more preferably, less than 10 microns as measured from the microprojection surface.

In a further embodiment of the invention, the formulation comprises a hydrogel which can be incorporated into a gel pack.

Correspondingly, in certain embodiments of the invention, the hydrogel formulations contain at least one immunologically active agent. Preferably, the agent comprises one of the aforementioned vaccines, including, without limitation, viruses and bacteria, protein-based vaccines, polysaccharide-based vaccine, and nucleic acid-based vaccines.

The hydrogel formulations preferably comprise water-based hydrogels having macromolecular polymeric networks.

In a preferred embodiment of the invention, the polymer network comprises, without limitation, hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics.

The hydrogel formulations preferably include one surfactant, which can be zwitterionic, amphoteric, cationic, anionic, or nonionic.

In one embodiment of the invention, the surfactant can comprise sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives such as sorbitan laureate, and alkoxylated alcohols such as laureth-4.

In another embodiment, the hydrogel formulations include polymeric materials or polymers having amphiphilic properties, which can comprise, without limitation, cellulose derivatives, such as hydroxyethylcellulose (HEC), hydroxypropyl-methylcellulose (HPMC), hydroxypropycellulose (HPC), methylcellulose (MC), hydroxyethylmethylcellulose (HEMC), or ethylhydroxyethylcellulose (EHEC), as well as pluronics.

In a further embodiment of the invention, the hydrogel formulations contain at least one pathway patency modulator, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, and EDTA.

In yet another embodiment of the invention, the hydrogel formulations include at least one vasoconstrictor, which can comprise, without limitation, epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline, and the mixtures thereof.

In a further aspect of the gel pack embodiments, the vaccine can be contained in a hydrogel formulation in the gel pack and in a biocompatible coating applied to the microprojection member.

In another embodiment of the invention, the ultrasonic device is adhered to the microprojection member.

In yet another embodiment of the invention, the ultrasonic device is adhered to a gel pack.

In another embodiment of the invention, the ultrasonic device further includes a matching layer to facilitate transfer of ultrasonic energy from the ultrasonic device to the microprojection member. Preferably, a double-sided adhesive layer is used to attach the ultrasonic device to the matching layer.

In currently preferred embodiments of the invention, the ultrasonic device generates sound waves having a frequency at least approximately 20 kHz.

In accordance with one embodiment of the invention, the method for delivering a vaccine (contained in the hydrogel formulation or contained in the biocompatible coating on the microprojection member or both) can be accomplished by the following steps: the microprojection member is initially applied to the patient's skin, preferably via an actuator, wherein the microprojections pierce the stratum corneum. The ultrasonic device is then applied on the applied microprojection member.

In an alternative embodiment, after application and removal of the microprojection member, the ultrasonic device is then placed on the patient's skin proximate the pre-treated area.

In another embodiment of the invention, the microprojection device is applied to the patient's skin, the gel pack having a vaccine-containing hydrogel formulation is then placed on top of the applied microprojection member, wherein the hydrogel formulation migrates into and through the microslits in the stratum corneum produced by the microprojections. The microprojection member and gel pack are then removed and the ultrasonic device is placed on the patient's skin proximate the effected area.

In an alternative embodiment, the ultrasonic device is placed on top of the applied microprojection member-gel pack assembly.

In embodiments of the invention wherein the formulation comprises a coating on the microprojection member, the step of transmitting ultrasonic energy with the ultrasonic device occurs preferably in the range of approximately 5 sec to 30 min after applying the microprojection member, and more preferably, in the range of approximately 30 sec to 15 min.

In embodiments of the invention wherein the formulation comprises a hydrogel, the step of transmitting ultrasonic energy with the ultrasonic device occurs preferably in the range of approximately 5 min to 24 h after applying the microprojection member, and more preferably, in the range of approximately 10 min to 4 h.

In embodiments of the invention wherein the formulation comprises a hydrogel incorporated in a gel pack and a coating on the microprojection member, the step of transmitting ultrasonic energy with the ultrasonic device occurs preferably in the range of approximately 5 sec to 24 h after applying the microprojection member, and more preferably, in the range of approximately 30 sec to 4 h.

Preferably, in the noted embodiments of the invention, the step of transmitting ultrasonic energy comprises applying sound waves having a frequency in the range of approximately 20 kHz to 10 MHz. More preferably, sound waves having a frequency in the range of approximately 20 kHz to 1 MHz are employed.

Also preferably, in the noted embodiments of the invention, the step of transmitting ultrasonic energy comprises applying energy having an intensity in the range of approximately 0.01 W/cm² to 100 W/cm². More preferably, energy having an intensity in the range of approximately 1 W/cm² to 20 W/cm² is employed.

In another aspect, the methods of the invention preferably comprise transmitting ultrasonic energy for a duration in the range of approximately 5 sec to 1 h and more preferably in the range of approximately 30 sec to 10 min.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of one embodiment of a transducer for an ultrasonic device for transdermally delivering a vaccine, according to the invention;

FIG. 2 is a perspective view of a portion of one example of a microprojection member;

FIG. 3 is a perspective view of the microprojection member shown in FIG. 2 having a coating deposited on the microprojections, according to the invention;

FIG. 3A is a cross-sectional view of a single microprojection taken along line 3A-3A in FIG. 3, according to the invention;

FIG. 4 is a side sectional view of a microprojection member having an adhesive backing;

FIG. 5 is a side sectional view of a retainer having a microprojection member disposed therein;

FIG. 6 is a perspective view of the retainer shown in FIG. 5;

FIG. 7 is an exploded perspective view of one embodiment of a gel pack of a microprojection system;

FIG. 8 is an exploded perspective view of one embodiment of a microprojection assembly that is employed in conjunction with the gel pack shown in FIG. 7; and

FIG. 9 is a perspective view of another embodiment of a microprojection system.

### DETAILED DESCRIPTION OF THE INVENTION

Before describing the present invention in detail, it is to be understood that this invention is not limited to particularly exemplified materials, methods or formulations as such may, of course, vary. Thus, although a number of materials, methods and formulations, similar or equivalent to those described herein, can be used in the practice of the present invention, the preferred materials, methods and formulations are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microprojection” includes two or more such microprojections and the like.

### Definitions

The term “transdermal”, as used herein, means the delivery of an agent into and/or through the skin for local or systemic therapy.

The term “transdermal flux”, as used herein, means the rate of transdermal delivery.

The term “vaccine”, as used herein, refers to a composition of matter or mixture containing an immunologically active agent or an agent, such as an antigen, which is capable of triggering a beneficial immune response when administered in an immunologically effective amount. Examples of such agents include, without limitation, viruses and bacteria, protein-based vaccines, polysaccharide-based vaccine, and nucleic acid-based vaccines.

Suitable antigenic agents that can be used in the present invention include, without limitation, antigens in the form of proteins, polysaccharide conjugates, oligosaccharides, and lipoproteins. These subunit vaccines include Bordetella pertussis (recombinant DPT vaccine —acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitis, pseudomonas aeruginosa, streptococcus pneumoniae, treponema pallidum, and vibrio cholerae, and mixtures thereof.

A number of commercially available vaccines, which contain antigenic agents also have utility with the present invention including, without limitation, flu vaccines, Lyme disease vaccine, rabies vaccine, measles vaccine, mumps vaccine, chicken pox vaccine, small pox vaccine, hepatitis vaccine, pertussis vaccine, and diptheria vaccine.

Vaccines comprising nucleic acids that can be delivered according to the methods of the invention, include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct. The antigen that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include aluminum phosphate gel; aluminum hydroxide; algal glucan: [β-glucan; cholera toxin B subunit; CRL1005: ABA block polymer with mean values of x=8 and y=205; gamma insulin: linear (unbranched) B-D(2->1) polyfructofuranoxyl-α-D-glucose; Gerbu adjuvant: N-acetylglucosamine-(β 14)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo [4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H108N₆O₁₉PNa—3H₂O (MTP); Murametide: Nac-Mur-L-Ala-D-Gln-OCH₃; Pleuran: β-glucan; QS-21; S-28463: 4-amino-a,a-dimethyl-1H-imidazo[4,5-c]quinoline-1-ethanol; sclavo peptide: VQGEESNDK.HCl (1L-1β 163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL4, IL10, gamma interferon, and NF kappa B regulatory signaling proteins can be used.

The noted vaccines can also be in various forms, such as free bases, acids, charged or uncharged molecules, components of molecular complexes or pharmaceutically acceptable salts. Further, simple derivatives of the active agents (such as ethers, esters, amides, etc.), which are easily hydrolyzed at body pH, enzymes, etc., can be employed.

It is to be understood that more than one vaccine may be incorporated into the agent source, reservoirs, and/or coatings of this invention, and that the use of the term “active agent” in no way excludes the use of two or more such active agents or drugs.

The term “biologically effective amount” or “biologically effective rate”, as used herein, means the vaccine is an immunologically active agent and refers to the amount or rate of the immunologically active agent needed to stimulate or initiate the desired immunologic, often beneficial result. The amount of the immunologically active agent employed in the hydrogel formulations and coatings of the invention will be that amount necessary to deliver an amount of the active agent needed to achieve the desired immunological result. In practice, this will vary widely depending upon the particular immunologically active agent being delivered, the site of delivery, and the dissolution and release kinetics for delivery of the active agent into skin tissues.

The term “microprojections”, as used herein, refers to piercing elements which are adapted to pierce or cut through the stratum corneum into the underlying epidermis layer, or epidermis and dermis layers, of the skin of a living animal, particularly a mammal and more particularly a human.

In one embodiment of the invention, the piercing elements have a projection length less than 1000 microns. In a further embodiment, the piercing elements have a projection length of less than 500 microns, more preferably, less than 250 microns. The microprojections typically have a width and thickness of about 5 to 50 microns. The microprojections may be formed in different shapes, such as needles, hollow needles, blades, pins, punches, and combinations thereof.

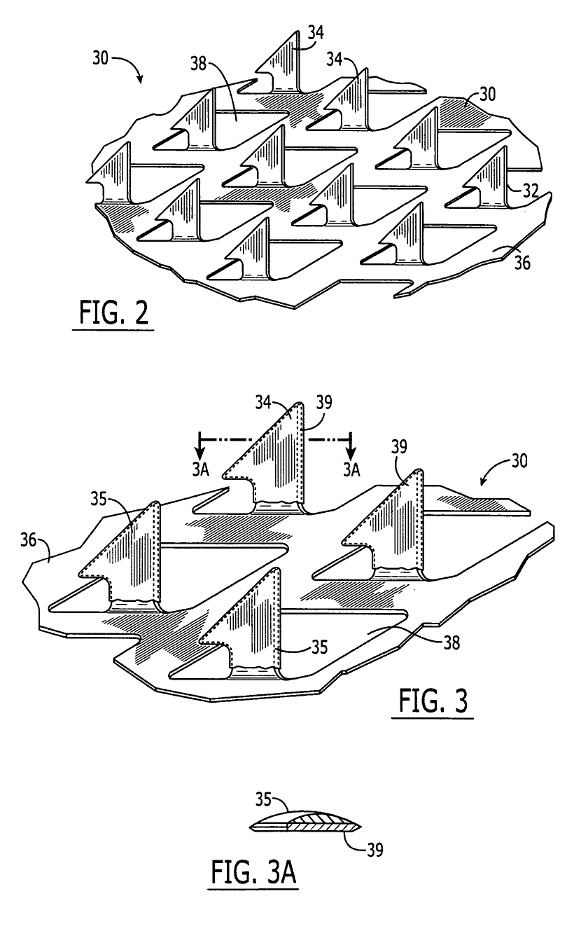

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration, such as that shown in FIG. 2. The microprojection member can also be formed in other known manners, such as by forming one or more strips having microprojections along an edge of each of the strip(s) as disclosed in U.S. Pat. No. 6,050,988, which is hereby incorporated by reference in its entirety.

The terms “ultrasound” and “ultrasonic”, as used herein, refers to ultrasonic waves or vibrations having a frequency above the human ear's audibility limit. As is well known in the art, such frequencies are typically greater than approximately 20,000 cycles/sec.

The term “ultrasound assisted”, as used herein, generally refers to the delivery of a therapeutic agent (charged, uncharged, or mixtures thereof), particularly a vaccine, through a body surface (such as skin, mucous membrane, or nails) wherein the delivery is at least partially induced or aided by the application of ultrasonic energy in the form(s) of high frequency sound waves and/or vibrations.

As indicated above, the present invention generally comprises (i) a microprojection member (or system) having a plurality of microprojections (or array thereof) that are adapted to pierce through the stratum corneum into the underlying epidermis layer, or epidermis and dermis layers and (ii) an ultrasonic device for transdermal delivery of biologically active agents.

In one embodiment, the microprojections have a coating thereon that contains at least one vaccine. Upon piercing the stratum corneum layer of the skin, the vaccine-containing coating is dissolved by body fluid (intracellular fluids and extracellular fluids such as interstitial fluid) and released into the skin for vaccination. As discussed in detail herein, after application of the microprojection member, ultrasound (i.e., ultrasonic frequency or waves) is applied to the member or the skin site in which the member was applied via the ultrasonic device to, among other things, enhance vaccine flux. Applicants have further found that the application of ultrasound increases cellular uptake of polypeptide-based vaccines and DNA vaccines to boost gene expression and immunity.

As is well known in the art, the application of ultrasound is typically accomplished by means of a transducer. As is also well known in the art, an ultrasound transducer produces ultrasound by converting electrical energy into mechanical energy.

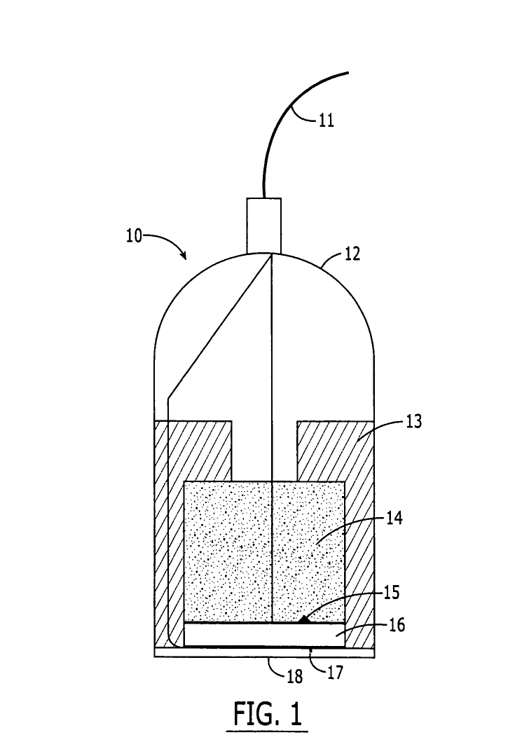

Referring now to FIG. 1 there is shown a schematic illustration of an exemplary transducer 10 for an ultrasonic device that can be used in accordance with the present invention. As illustrated in FIG. 1, the transducer 10 generally includes a coaxial cable 11, housing 12, acoustic insulator 13, backing block 14, live electrode 15, piezoelectric crystal 16, grounded electrode 17 and matching layer 18.

The front and back faces of the disk-shaped piezoelectric crystal 16 are typically coated with a thin film to ensure good contact with the two electrodes 15, 17 that supply the electric voltage that causes the crystal 16 to vibrate.

The front electrode is earthed to protect the patient from electric shock, and is also covered by the matching layer 18, which improves the transmission of the ultrasonic energy into the body.

Optionally, the matching layer 18 is covered with a disposable double-sided adhesive layer that further improves contact between the transducer 10 and the gel pack (e.g., 60), or the microprojection member (e.g., 70), or the skin. According to the invention, a new disposable double-sided adhesive is adhered to the matching layer 18 prior every single use.

As discussed in detail herein, following microprojection array application to the skin, the transducer 10 is adhered to the gel pack (or the microprojection member, or the skin, depending on the system configuration used) and the ultrasound treatment is applied. In an alternative embodiment, the matching layer 18 is replaced with the disposable double-sided adhesive. In yet a further alternative embodiment, the double sided adhesive is an integral part of the gel pack or the microprojection member.

As illustrated in FIG. 1, the back face of the crystal 16 abuts a thick backing block 14. The backing block 14 is adapted to absorb the ultrasound transmitted into the transducer 10 and dampen the vibration of the crystal 16 (thereby reducing the spatial pulse length in pulsed ultrasound transmission).

Finally, the acoustic insulator 13, which typically comprises cork or rubber, prevents the ultrasound from passing into the plastic housing 12.

As will be appreciated by one having ordinary skill in the art, various transducers and, hence, ultrasonic devices can be employed within the scope of the invention to provide the ultrasound or ultrasonic energy to enhance the vaccine flux.

According to the invention, the ultrasonic device can be employed with various microprojection members and systems to enhance the agent flux. Referring now to FIG. 2, there is shown one embodiment of a microprojection member 30 for use with the present invention. As illustrated in FIG. 2, the microprojection member 30 includes a microprojection array 32 having a plurality of microprojections 34. The microprojections 34 preferably extend at substantially a 90° angle from the sheet 36, which in the noted embodiment includes openings 38.

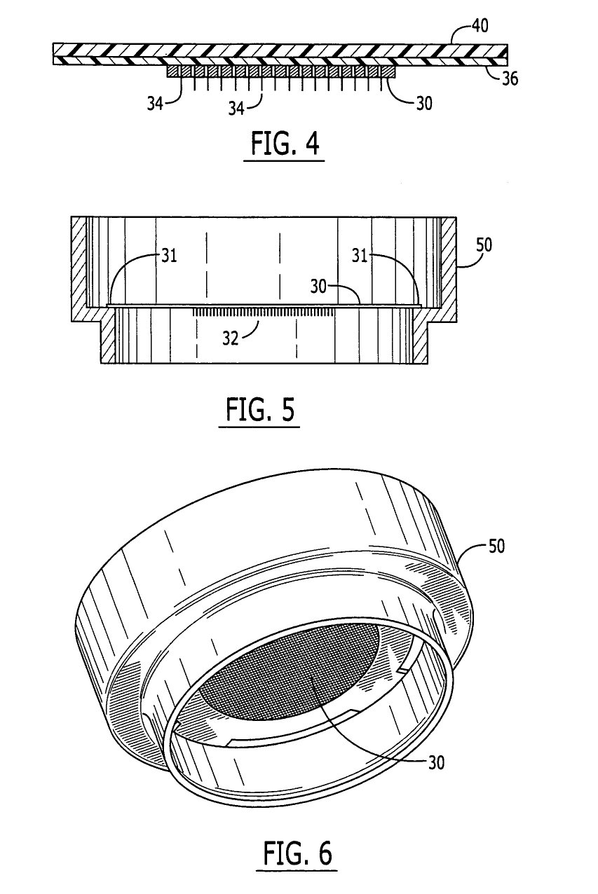

According to the invention, the sheet 36 may be incorporated into a delivery patch, including a backing 40 for the sheet 36, and may additionally include adhesive 16 for adhering the patch to the skin (see FIG. 4). In this embodiment, the microprojections 34 are formed by etching or punching a plurality of microprojections 34 from a thin metal sheet 36 and bending the microprojections 34 out of the plane of the sheet 36.

In one embodiment of the invention, the microprojection member 30 has a microprojection density of at least approximately 10 microprojections/cm², more preferably, in the range of at least approximately 200-2000 microprojections/cm². Preferably, the number of openings per unit area through which the agent passes is at least approximately 10 openings/cm² and less than about 2000 openings/cm².

As indicated, the microprojections 34 preferably have a projection length less than 1000 microns. In one embodiment, the microprojections 34 have a projection length of less than 500 microns, more preferably, less than 250 microns. The microprojections 34 also preferably have a width and thickness of about 5 to 50 microns.

The microprojection member 30 can be manufactured from various metals, such as stainless steel, titanium, nickel titanium alloys, or similar biocompatible materials, such as polymeric materials. Preferably, the microprojection member 30 is manufactured out of titanium.

According to the invention, the microprojection member 30 can also be constructed out of a non-conductive material, such as a polymer. Alternatively, the microprojection member can be coated with a non-conductive material, such as parylene.

Microprojection members that can be employed with the present invention include, but are not limited to, the members disclosed in U.S. Pat. Nos. 6,083,196, 6,050,988 and 6,091,975, which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

According to the invention, the biologically active agent (i.e., vaccine) to be delivered can be contained in the hydrogel formulation disposed in a gel pack reservoir (discussed in detail below), contained in a biocompatible coating that is disposed on the microprojection member 30 or contained in both the hydrogel formulation and the biocompatible coating.

Referring now to FIG. 3, there is shown a microprojection member 30 having microprojections 34 that include a biocompatible coating 35. According to the invention, the coating 35 can partially or completely cover each microprojection 34. For example, the coating 35 can be in a dry pattern coating on the microprojections 34. The coating 35 can also be applied before or after the microprojections 34 are formed.

According to the invention, the coating 35 can be applied to the microprojections 34 by a variety of known methods. Preferably, the coating is only applied to those portions the microprojection member 30 or microprojections 34 that penetrate the skin (e.g., tips 39).

One such coating method comprises dip-coating. Dip-coating can be described as a means to coat the microprojections by partially or totally immersing the microprojections 34 into a coating solution. By use of a partial immersion technique, it is possible to limit the coating 35 to only the tips 39 of the microprojections 34.

A further coating method comprises roller coating, which employs a roller coating mechanism that similarly limits the coating 35 to the tips 39 of the microprojections 34. The roller coating method is disclosed in U.S. application Ser. No. 10/099,604 (Pub. No. 2002/0132054), which is incorporated by reference herein in its entirety.

As discussed in detail in the noted application, the disclosed roller coating method provides a smooth coating that is not easily dislodged from the microprojections 34 during skin piercing. The smooth cross-section of the microprojection tip coating 35 is further illustrated in FIG. 3A.

According to the invention, the microprojections 34 can further include means adapted to receive and/or enhance the volume of the coating 35, such as apertures (not shown), grooves (not shown), surface irregularities (not shown) or similar modifications, wherein the means provides increased surface area upon which a greater amount of coating can be deposited.

Another coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 10 and then dried.

Pattern coating can also be employed to coat the microprojections 34. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

As indicated, according to one embodiment of the invention, the coating formulations applied to the microprojection member 30 to form solid coatings can comprise aqueous and non-aqueous formulations having at least one vaccine. According to the invention, the vaccine can be dissolved within a biocompatible carrier or suspended within the carrier.

The vaccine preferably includes, without limitation, viruses and bacteria, protein-based vaccines, polysaccharide-based vaccine, and nucleic acid-based vaccines.

Suitable antigenic agents include, without limitation, antigens in the form of proteins, polysaccharide conjugates, oligosaccharides, and lipoproteins. These subunit vaccines include Bordetella pertussis (recombinant DPT vaccine—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitis, pseudomonas aeruginosa, streptococcus pneumoniae, treponema pallidum, and vibrio cholerae, and mixtures thereof.

Additional commercially available vaccines, which contain antigenic agents, include, without limitation, flu vaccines, Lyme disease vaccine, rabies vaccine, measles vaccine, mumps vaccine, chicken pox vaccine, small pox vaccine, hepatitis vaccine, pertussis vaccine, and diptheria vaccine.

Vaccines comprising nucleic acids include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, MRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct. The antigen that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include aluminum phosphate gel; aluminum hydroxide; algal glucan: β-glucan; cholera toxin B subunit; CRL1005: ABA block polymer with mean values of x=8 and y=205; gamma insulin: linear (unbranched) 13-D(2->1) polyfructofuranoxyl-α-D-glucose; Gerbu adjuvant: N-acetylglucosamine-(p 14)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa—3H₂O (MTP); Murametide: Nac-Mur-L-Ala-D-Gln-OCH₃; Pleuran: β-glucan; QS-21; S-28463: 4-amino-a, a-dimethyl-1H-imidazo[4,5-c]quinoline-1-ethanol; sclavo peptide: VQGEESNDK.HCl (1L-1β 163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-1 8, IL-2 IL-12, IL-15, IL4, IL10, gamma interferon, and NF kappa B regulatory signaling proteins can be used.

The noted vaccines can be in various forms, such as free bases, acids, charged or uncharged molecules, components of molecular complexes or pharmaceutically acceptable salts. Further, simple derivatives of the active agents (such as ethers, esters, amides, etc.), which are easily hydrolyzed at body pH, enzymes, etc., can be employed.

According to the invention, the coating formulations preferably include at least one wetting agent. As is well known in the art, wetting agents can generally be described as amphiphilic molecules. When a solution containing the wetting agent is applied to a hydrophobic substrate, the hydrophobic groups of the molecule bind to the hydrophobic substrate, while the bydrophilic portion of the molecule stays in contact with water. As a result, the hydrophobic surface of the substrate is not coated with hydrophobic groups of the wetting agent, making it susceptible to wetting by the solvent. Wetting agents include surfactants as well as polymers presenting amphiphillic properties.

In one embodiment of the invention, the coating formulations include at least one surfactant. According to the invention, the surfactant(s) can be zwitterionic, amphoteric, cationic, anionic, or nonionic. Examples of surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laureate, and alkoxylated alcohols such as laureth-4. Most preferred surfactants include Tween 20, Tween 80, and SDS.

Preferably, the concentration of the surfactant is in the range of approximately 0.001-2 wt. % of the coating solution formulation.

In a further embodiment of the invention, the coating formulations include at least one polymeric material or polymer that has amphiphilic properties. Examples of the noted polymers include, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties is preferably in the range of approximately 0.01-20 wt. %, more preferably, in the range of approximately 0.03-10 wt. % of the coating formulation. Even more preferably, the concentration of the wetting agent is in the range of approximately 0.1-5 wt. % of the coating formulation.

As will be appreciated by one having ordinary skill in the art, the noted wetting agents can be used separately or in combinations.

According to the invention, the coating formulations can further include a hydrophilic polymer. Preferably the hydrophilic polymer is selected from the following group: poly(vinyl alcohol), poly(ethylene oxide), poly(2-hydroxyethylmethacrylate), poly(n-vinyl pyrolidone), polyethylene glycol and mixtures thereof, and like polymers. As is well known in the art, the noted polymers increase viscosity.

The concentration of the hydrophilic polymer in the coating formulation is preferably in the range of approximately 0.01-20 wt. %, more preferably, in the range of approximately 0.03-10 wt. % of the coating formulation. Even more preferably, the concentration of the wetting agent is in the range of approximately 0.1-5 wt. % of the coating formulation.

According to the invention, the coating formulations can further include a biocompatible carrier such as those disclosed in Co-Pending U.S. application Ser. No. 10/127,108, which is incorporated by reference herein in its entirety. Examples of biocompatible carriers include human albumin, bioengineered human albumin, polyglutamic acid, polyaspartic acid, polyhistidine, pentosan polysulfate, polyamino acids, sucrose, trehalose, melezitose, raffinose and stachyose.

The concentration of the biocompatible carrier in the coating formulation is preferably in the range of approximately 2-70 wt. %, more preferably, in the range of approximately 5-50 wt. % of the coating formulation. Even more preferably, the concentration of the wetting agent is in the range of approximately 10-40 wt. % of the coating formulation.

The coatings of the invention can further include a vasoconstrictor such as those disclosed in Co-Pending U.S. application Ser. Nos. 10/674,626 and 60/514,433, which are incorporated by reference herein in their entirety. As set forth in the noted Co-Pending Applications, the vasoconstrictor is used to control bleeding during and after application on the microprojection member. Preferred vasoconstrictors include, but are not limited to, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating.

In yet another embodiment of the invention, the coating formulations include at least one “pathway patency modulator”, such as those disclosed in Co-Pending U.S. application Ser. No. 09/950,436, which is incorporated by reference herein in its entirety. As set forth in the noted Co-Pending Application, the pathway patency modulators prevent or diminish the skin's natural healing processes thereby preventing the closure of the pathways or microslits formed in the stratum corneum by the microprojection member array. Examples of pathway patency modulators include, without limitation, osmotic agents (e.g., sodium chloride), and zwitterionic compounds (e.g., amino acids).

The term “pathway patency modulator,” as defined in the Co-Pending Application, further includes anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In another embodiment of the invention, the coating formulation includes at least one antioxidant, which can be sequestering, such as sodium citrate, citric acid, EDTA (ethylene-dinitrilo-tetraacetic acid), or free radical scavengers, such as ascorbic acid, methionine, sodium ascorbate, and the like. Presently preferred antioxidants include EDTA and methionine.

In certain embodiments of the invention, the viscosity of the coating formulation is enhanced by adding low volatility counterions. In one embodiment, the agent has a positive charge at the formulation pH and the viscosity-enhancing counterion comprises an acid having at least two acidic pKas. Suitable acids include maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, aspartic acid, glutamic acid, carbonic acid, sulfuric acid, and phosphoric acid.

Another preferred embodiment is directed to a viscosity-enhancing mixture of counterions wherein the agent has a positive charge at the formulation pH and at least one of the counterion is an acid having at least two acidic pKas. The other counterion is an acid with one or more pKas. Examples of suitable acids include hydrochloric acid, hydrobromic acid, nitric acid, sulfuric acid, maleic acid, phosphoric acid, benzene sulfonic acid, methane sulfonic acid, citric acid, succinic acid, glycolic acid, gluconic acid, glucuronic acid, lactic acid, malic acid, pyruvic acid, tartaric acid, tartronic acid, fumaric acid, acetic acid, propionic acid, pentanoic acid, carbonic acid, malonic acid, adipic acid, citraconic acid, levulinic acid, glutaric acid, itaconic acid, meglutol, mesaconic acid, citramalic acid, citric acid, aspartic acid, glutamic acid, tricarballylic acid and ethylenediaminetetraacetic acid.

Generally, in the noted embodiments of the invention, the amount of counterion should neutralize the charge of the antigenic agent. In such embodiments, the counterion or the mixture of counterion is present in amounts necessary to neutralize the charge present on the agent at the pH of the formulation. Excess of counterion (as the free acid or as a salt) can be added to the formulation in order to control pH and to provide adequate buffering capacity.

In another preferred embodiment, the agent has a positive charge and the counterion is a viscosity-enhancing mixture of counterions chosen from the group of citric acid, tartaric acid, malic acid, hydrochloric acid, glycolic acid, and acetic acid. Preferably, counterions are added to the formulation to achieve a viscosity in the range of about 20-200 cp.

In a preferred embodiment, the viscosity-enhancing counterion is an acidic counterion such as a low volatility weak acid. Low volatility weak acid counterions present at least one acidic pKa and a melting point higher than about 50° C. or a boiling point higher than about 170° C. at Pₐₜₘ. Examples of such acids include citric acid, succinic acid, glycolic acid, gluconic acid, glucuronic acid, lactic acid, malic acid, pyruvic acid, tartaric acid, tartronic acid, and fumaric acid.

In another preferred embodiment the counterion is a strong acid. Strong acids can be defined as presenting at least one pKa lower than about 2. Examples of such acids include hydrochloric acid, hydrobromic acid, nitric acid, sulfonic acid, sulfuric acid, maleic acid, phosphoric acid, benzene sulfonic acid and methane sulfonic acid.

Another preferred embodiment is directed to a mixture of counterions wherein at least one of the counterion is a strong acid and at least one of the counterion is a low volatility weak acid.

Another preferred embodiment is directed to a mixture of counterions wherein at least one of the counterions is a strong acid and at least one of the counterion is a weak acid with high volatility. Volatile weak acid counterions present at least one pKa higher than about 2 and a melting point lower than about 50° C. or a boiling point lower than about 170° C. at Pₐₜₘ. Examples of such acids include acetic acid, propionic acid, pentanoic acid and the like.

Preferably, the acidic counterion is present in amounts necessary to neutralize the positive charge present on the antigenic agent at the pH of the formulation. Excess of counterion (as the free acid or as a salt) can be added to the formulation in order to control pH and to provide adequate buffering capacity.

In yet other embodiments of the invention, particularly where the antigenic agent has a negative charge, the coating formulation further comprises a low volatility basic counter ion.

In a preferred embodiment, the coating formulation comprises a low volatility weak base counterion. Low volatility weak bases present at least one basic pKa and a melting point higher than about 50° C. or a boiling point higher than about 1 70° C. at Pₐₜₘ. Examples of such bases include monoethanolomine, diethanolamine, triethanolamine, tromethamine, methylglucamine, and glucosamine.

In another embodiment, the low volatility counterion comprises a basic zwitterions presenting at least one acidic pKa, and at least two basic pKa's, wherein the number of basic pKa's is greater than the number of acidic pkA's. Examples of such compounds include histidine, lysine, and arginine.

In yet other embodiments, the low volatility counterion comprises a strong base presenting at least one pKa higher than about 12. Examples of such bases include sodium hydroxide, potassium hydroxide, calcium hydroxide, and magnesium hydroxide.

Other preferred embodiments comprise a mixture of basic counterions comprising a strong base and a weak base with low volatility. Alternatively, suitable counterions include a strong base and a weak base with high volatility. High volatility bases present at least one basic pKa lower than about 12 and a melting point lower than about 50° C. or a boiling point lower than about 170° C. at Pₐₜₘ. Examples of such bases include ammonia and morpholine.

Preferably, the basic counterion is present in amounts necessary to neutralize the negative charge present on the antigenic agent at the pH of the formulation. Excess of counterion (as the free base or as a salt) can be added to the formulation in order to control pH and to provide adequate buffering capacity.

According to the invention, the coating formulations can also include a non-aqueous solvent, such as ethanol, chloroform, ether, propylene glycol, polyethylene glycol and the like, dyes, pigments, inert fillers, permeation enhancers, excipients, and other conventional components of pharmaceutical products or transdermal devices known in the art.

Other known formulation additives can also be added to the coating formulations as long as they do not adversely affect the necessary solubility and viscosity characteristics of the coating formulation and the physical integrity of the dried coating.

Preferably, the coating formulations have a viscosity less than approximately 500 centipoise and greater than 3 centipoise in order to effectively coat each microprojection 10. More preferably, the coating formulations have a viscosity in the range of approximately 3-200 centipoise.

According to the invention, the desired coating thickness is dependent upon the density of the microprojections per unit area of the sheet and the viscosity and concentration of the coating composition as well as the coating method chosen. Preferably, the coating thickness is less than 50 microns.

In one embodiment, the coating thickness is less than 25 microns, more preferably, less than 10 microns as measured from the microprojection surface. Even more preferably, the coating thickness is in the range of approximately 1 to 10 microns.

In all cases, after a coating has been applied, the coating formulation is dried onto the microprojections 10 by various means. In a preferred embodiment of the invention, the coated member is dried in ambient room conditions. However, various temperatures and humidity levels can be used to dry the coating formulation onto the microprojections. Additionally, the coated member can be heated, lyophilized, freeze dried or similar techniques used to remove the water from the coating.

Referring now to FIGS. 5 and 6, for storage and application (in accordance with one embodiment of the invention), the microprojection member 30 is preferably suspended in a retainer ring 50 by adhesive tabs 31, as described in detail in Co-Pending U.S. application Ser. No. 09/976,762 (Pub. No. 2002/0091357), which is incorporated by reference herein in its entirety.

After placement of the microprojection member 30 in the retainer ring 50, the microprojection member 30 is applied to the patient's skin. Preferably, the microprojection member 30 is applied to the skin using an impact applicator, such as disclosed in Co-Pending U.S. application Ser. No. 09/976,798, which is incorporated by reference herein in its entirety.

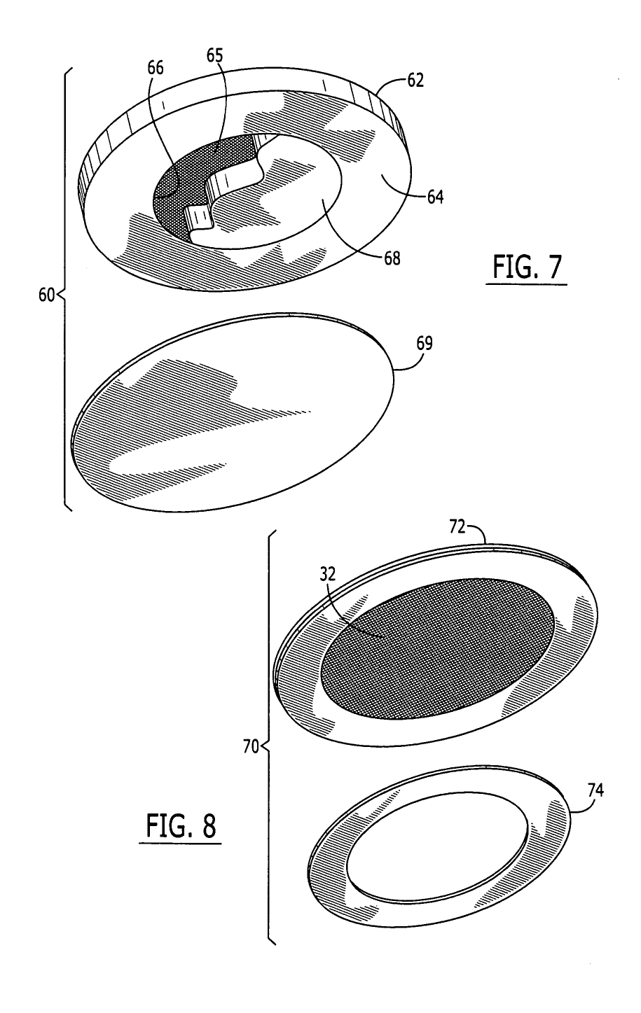

Referring now to FIGS. 7 and 8, there is shown a further microprojection system that can be employed within the scope of the present invention. As illustrated in FIGS. 7 and 8, the system 60 includes a gel pack 62 and a microprojection assembly 70, having a microprojection member, such as the microprojection member 30 shown in FIG. 2.

According to the invention, the gel pack 62 includes a housing or ring 64 having a centrally disposed reservoir or opening 66 that is adapted to receive a predetermined amount of a hydrogel formulation 68 therein. As illustrated in FIG. 7, the ring 64 further includes a backing member 65 that is disposed on the outer planar surface of the ring 64. Preferably, the backing member 65 is impermeable to the hydrogel formulation.

In a preferred embodiment, the gel pack 60 further includes a strippable release liner 69 that is adhered to the outer surface of the gel pack ring 64 via a conventional adhesive. As described in detail below, the release liner 69 is removed prior to application of the gel pack 60 to the applied (or engaged) microprojection assembly 70.

Referring now to FIG. 8, the microprojection assembly 70 includes a backing membrane ring 72 and a similar microprojection array 32. The microprojection assembly further includes a skin adhesive ring 74.

Further details of the illustrated gel pack 60 and microprojection assembly 70, as well as additional embodiments thereof that can be employed within the scope of the present invention are set forth in Co-Pending Application No. 60/514,387, which is incorporated by reference herein in its entirety.

As indicated above, in at least one embodiment of the invention, the hydrogel formulation contains at least one biologically active agent, preferably a vaccine. In an alternative embodiment of the invention, the hydrogel formulation is devoid of a vaccine and, hence, is merely a hydration mechanism.

According to the invention, when the hydrogel formulation is devoid of a vaccine, the vaccine is either coated on the microprojection array 32, as described above, or contained in a solid film, such as disclosed in PCT Pub. No. WO 98/28037, which is similarly incorporated by reference herein in its entirety, on the skin side of the microprojection array 32, such as disclosed in the noted Co-Pending Application No. 60/514,387 or the top surface of the array 32.

As discussed in detail in the Co-Pending Application, the solid film is typically made by casting a liquid formulation consisting of the vaccine, a polymeric material, such as hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), or pluronics, a plasticising agent, such as glycerol, propylene glycol, or polyethylene glycol, a surfactant, such as Tween 20 or Tween 80, and a volatile solvent, such as water, isopropanol, or ethanol. Following casting and subsequent evaporation of the solvent, a solid film is produced.

Preferably, the hydrogel formulations of the invention comprise water-based hydrogels. Hydrogels are preferred formulations because of their high water content and biocompatibility.

As is well known in the art, hydrogels are macromolecular polymeric networks that are swollen in water. Examples of suitable polymeric networks include, without limitation, hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics. The most preferred polymeric materials are cellulose derivatives. These polymers can be obtained in various grades presenting different average molecular weight and therefore exhibit different rheological properties.

Preferably, the concentration of the polymeric material is in the range of approximately 0.5-40 wt. % of the hydrogel formulation.

The hydrogel formulations of the invention preferably have sufficient surface activity to insure that the formulations exhibit adequate wetting characteristics, which are important for establishing optimum contact between the formulation and the microprojection array 32 and skin and, optionally, the solid film.

According to the invention, adequate wetting properties are achieved by incorporating a wetting agent in the hydrogel formulation. Optionally, a wetting agent can also be incorporated in the solid film.

Preferably the wetting agents include at least one surfactant. According to the invention, the surfactant(s) can be zwitterionic, amphoteric, cationic, anionic, or nonionic. Examples of surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laureate, and alkoxylated alcohols such as laureth-4. Most preferred surfactants include Tween 20, Tween 80, and SDS.

Preferably, the wetting agents also include polymeric materials or polymers having amphiphilic properties. Examples of the noted polymers include, without limitation, cellulose derivatives, such as hydroxyethylcellulose (HEC), hydroxypropyl-methylcellulose (HPMC), hydroxypropycellulose (HPC), methylcellulose (MC), hydroxyethylmethylcellulose (HEMC), or ethylhydroxyethylcellulose (EHEC), as well as pluronics.

Preferably, the concentration of the surfactant is in the range of approximately 0.001-2 wt. % of the hydrogel formulation. The concentration of the polymer that exhibits amphiphilic properties is preferably in the range of approximately 0.5-40 wt. % of the hydrogel formulation.

As will be appreciated by one having ordinary skill in the art, the noted wetting agents can be used separately or in combinations.

According to the invention, the hydrogel formulations can similarly include at least one pathway patency modulator or “anti-healing agent”, such as those disclosed in Co-Pending U.S. application Ser. No. 09/950,436. As stated above, the pathway patency modulators include, without limitation, osmotic agents (e.g., sodium chloride), and zwitterionic compounds (e.g., amino acids). The pathway patency modulators also include anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinate sodium salt, paramethasone disodium phosphate and prednisolone 21-succinate sodium salt, and anticoagulants, such as citric acid, citrate salts (e.g., sodium citrate), dextran sulfate sodium, and EDTA.

The hydrogel formulation can further include at least one vasoconstrictor. As stated, suitable vasoconstrictors include, without limitation, epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline, and the mixtures thereof.

According to the invention, the hydrogel formulations can also include a non-aqueous solvent, such as ethanol, propylene glycol, polyethylene glycol and the like, dyes, pigments, inert fillers, permeation enhancers, excipients, and other conventional components of pharmaceutical products or transdermal devices known in the art.

The hydrogel formulations of the invention exhibit adequate viscosity so that the formulation can be contained in the gel pack 60, keeps its integrity during the application process, and is fluid enough so that it can flow through the microprojection assembly openings 380 and into the skin pathways.

For hydrogel formulations that exhibit Newtonian properties, the viscosity of the hydrogel formulation is preferably in the range of approximately 2-30 Poises (P), as measured at 25° C. For shear-thinning hydrogel formulations, the viscosity, as measured at 25° C., is preferably in the range of 1.5-30 P or 0.5 and 10 P, at shear rates of 667/s and 2667/s, respectively. For dilatant formulations, the viscosity, as measured at 25° C., is preferably in the range of approximately 1.5-30 P, at a shear rate of 667/s.

As indicated, in at least one embodiment of the invention, the hydrogel formulation contains at least one vaccine. Preferably, the vaccine comprises one of the aforementioned vaccines.

According to the invention, when the hydrogel formulation contains one of the aforementioned vaccines, the vaccine can be present at a concentration in excess of saturation or below saturation. The amount of a vaccine employed in the microprojection system will be that amount necessary to deliver a therapeutically effective amount of the vaccine to achieve the desired result. In practice, this will vary widely depending upon the particular vaccine, the site of delivery, the severity of the condition, and the desired therapeutic effect. Thus, it is not practical to define a particular range for the therapeutically effective amount of a vaccine incorporated into the method.

In one embodiment of the invention, the concentration of the vaccine is in the range of at least 1-40 wt. % of the hydrogel formulation.

For storage and application, the microprojection assembly is similarly preferably suspended in the retainer 50 shown in FIGS. 5 and 6. After placement of the microprojection assembly 70 in the retainer 50, the microprojection assembly 70 is applied to the patient's skin. Preferably, the microprojection assembly 70 is similarly applied to the skin using an impact applicator, such as disclosed in Co-Pending U.S. application Ser. No. 09/976,798.

After application of the microprojection assembly 70, the release liner 69 is removed from the gel pack 60. The gel pack 60 is then placed on the microprojection assembly 70, whereby the hydrogel formulation 68 is released from the gel pack 60 through the openings 38 in the microprojection array 32, passes through the microslits in the stratum corneum formed by the microprojections 34, migrates down the outer surfaces of the microprojections 34 and through the stratum corneum to achieve local or systemic therapy.

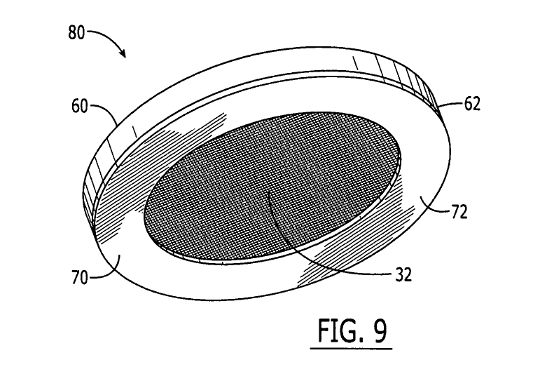

Referring now to FIG. 9, there is shown another embodiment of a microprojection system 80 that can be employed within the scope of the present invention. As illustrated in FIG. 9, the system comprises an integrated unit comprising the microprojection member 70 and gel pack 60 described above and shown in FIGS. 7 and 8.

In accordance with one embodiment of the invention, the method for delivering a vaccine (contained in the hydrogel formulation or contained in the biocompatible coating on the microprojection member or both) can be accomplished by the following steps: the coated microprojection member (e.g., 70) is initially applied to the patient's skin via an actuator wherein the microprojections 34 pierce the stratum corneum. The ultrasonic device is then applied on the applied microprojection member.

In an alternative embodiment, after application and removal of the coated microprojection member, the ultrasonic device is then placed on the patient's skin proximate the pre-treated area.

In another embodiment of the invention, the microprojection device 70 is applied to the patient's skin, the gel pack 60 having a vaccine-containing hydrogel formulation is then placed on top of the applied microprojection member 70, wherein the hydrogel formulation 68 migrates into and through the microslits in the stratum corneum produced by the microprojections 34. The microprojection member 70 and gel pack 60 are then removed and the ultrasonic device is placed on the patient's skin proximate the effected area.

In an alternative embodiment, the ultrasonic device is placed on top of the applied microprojection member-gel pack assembly 80.

In a further aspect of the gel pack embodiments, the vaccine is contained in hydrogel formulation in the gel pack 60 and in a biocompatible coating applied to the microprojection member 70.

Preferably, when a vaccine-coated microprojection array is used to practice the invention, the ultrasound treatment is applied 5 sec to 30 min after the initial application to the skin of the vaccine-coated microprojection array. More preferably, the ultrasound treatment is applied 30 sec to 15 min after the initial application to the skin of the vaccine-coated microprojection array.

Preferably, when a gel reservoir-containing vaccine is used to practice the invention, the ultrasound treatment is applied 5 min to 24 h after the initial application to the skin of the gel reservoir-containing vaccine. More preferably, the ultrasound treatment is applied 10 min to 4 h after application to the skin of the gel reservoir-containing vaccine.

Preferably, when the combination of a vaccine-coated microprojection array and a gel reservoir-containing vaccine is used to practice the invention, the ultrasound treatment is applied 5 sec to 24 h after the initial application to the skin of the combination of a vaccine-coated microprojection array and a gel reservoir-containing vaccine. More preferably, the ultrasound treatment is applied 30 sec to 4 h after the initial application to the skin of the combination of a vaccine-coated microprojection array and a gel reservoir-containing vaccine.

Preferably, the ultrasonic device applies sound waves having a frequency in the range of approximately 20 kHz to 10 MHz, more preferably, in the range of approximately 20 kHz -1 MHz.

Preferably, the applied intensities are in the range of approximately 0.01-100 W/cm². More preferably, the applied intensities are in the range of approximately 1-20 W/cm².

Preferably, the ultrasound treatment is applied for a duration in the range of approximately 5 sec to 1 h. More preferably, for a duration in the range of approximately 30 sec to 10 min.

### EXAMPLES

### Example 1

Preliminary experiments have demonstrated that microprojection array technology delivers DNA into skin, but gene expression and immune responses to encoded antigens were found to be low to not detectable. In this example we combine transdermal DNA vaccine delivery by microprojection array technology, using dry coated arrays or gel reservoirs, with ultrasound to assist intracellular DNA delivery. Immune responses to an expression vector encoding Hepatitis B virus surface antigen (HBsAg) are monitored. Nine treatment groups are evaluated:

Group 1: DNA-coated microprojection array (MA) delivery (2 min application time) without any augmentation of intracellular delivery.

Group 2: DNA-coated microprojection array delivery (2 min application time) followed by ultrasound after removal of the microprojection array.

Group 3: DNA-coated microprojection array delivery (1 min application time) followed by ultrasound with microprojection array remaining in place during ultrasound.

Group 4: Application of uncoated microprojection array followed by ultrasound with DNA in gel reservoir after removal of the microprojection array. The gel reservoir is in place for 15 min prior to ultrasound.

Group 4A: Application of uncoated microprojection array with DNA in gel reservoir after removal of the microprojection array, no ultrasound. The gel reservoir is in place for 16 min.

Group 5: Application of uncoated microprojection array followed by ultrasound with DNA in gel reservoir with microprojection array remaining in place during ultrasound. The gel reservoir is in place for 15 min prior to ultrasound.

Group 5A: Application of uncoated microprojection array with DNA in gel reservoir with microprojection array remaining in place, no ultrasound. The gel reservoir is in place for 16 min.

Group 6: topical DNA application followed by ultrasound 15 min after application.

Group 6A: topical DNA application for 16 min, no ultrasound.

### Materials and Methods

Microprojection arrays: MA 1035 (microprojection length 225 μm, 675 microprojections/cm², 2 cm² array) coated with pCMV-S (HBsAg expression plasmid—Aldevron, Fargo, N.Dak.).

Microprojection array coating: 60 μg DNA per array, obtained by roller coater methodology using an aqueous formulation containing 12 mg/mL plasmid, 12 mg/mL sucrose, and 2 mg/mL Tween 20.

DNA gel: 350 μL of an aqueous formulation containing 1.5% HEC, 3.6 mg/ml DNA, and 2 mg/mL Tween 20.

Topical DNA application: 50 μg DNA in 50 μl saline.

Ultrasound conditions: 1 MHz; 1 W/cm²; 1 minute, delivered by transducer described in FIG. 1.

DNA delivery to hairless guinea pig (HGP) skin: Microprojection array are applied to live HGP for 1 minute and the application site is marked. DNA delivery by microprojection array/DNA gel is augmented as indicated in the treatment table. Ultrasound is done immediately following DNA delivery by microprojection array, while all animals remain under anesthesia.

Humoral immune responses two weeks after one booster application at week four are measured using the ABBOTT AUSAB EIA Diagnostic Kit and quantification panel. Antibody titers of higher than the protective level of 10 mIU/ml are marked as “positive” in Table 1.

Cellular responses are determined using a surrogate assay to predict CTL activity: spleen cells are harvested at the time of obtaining the sera for antibody titer determination and the number of gamma interferon producing CD8 cells—after depletion of CD4 positive cells by anti-CD4-coated Dynabeads (Dynal, N.Y.)—are determined by ELISPOT assay after a five day in vitro re-stimulation with the HBsAg protein (Aldevron). A “positive” response is scored when (i) mean number of cells in wells re-stimulated with HBsAg are significantly (P<0.05, student's t test) higher than in wells re-stimulated with ovalbumin (Ova), an irrelevant antigen (ii) net number of spot forming cells (SFCs) (SFCs in wells stimulated with HBsAg minus number of SFCs in wells stimulated with Ova) is 5 or larger, and (iii) the ratio of mean number of SFCs in HBsAg wells to mean number of SFCs in Ova wells is greater than 2.0.

TABLE 1
Treatment Table and Immune Responses

| | | | Augmentation | Immune response | |
|---|---|---|---|---|---|
| Grp | n | DNA Delivery to Skin | Method | humoral | cellular |
| 1 | 4 | Coated MA | none | negative | negative |
| 2 | 4 | Coated MA, removed | ultrasound | positive | positive |
| 3 | 4 | Coated MA, integrated | ultrasound | positive | positive |
| 4 | 4 | Uncoated MA, removed, DNA gel | ultrasound | positive | positive |
| 4A | 4 | Uncoated MA, removed, DNA gel | none | negative | negative |
| 5 | 4 | Uncoated MA, integrated, DNA gel | ultrasound | positive | positive |
| 5A | 4 | Uncoated MA, integrated, DNA gel | none | negative | negative |
| 6 | 4 | Topical DNA | ultrasound | negative | negative |
| 6A | 4 | Topical DNA | none | negative | negative |

This example demonstrates that ultrasound can augment intracellular DNA uptake after delivery to skin by microprojection array or gel reservoir through microprojection array generated passages and can result in the induction of cellular and humoral immune responses to the antigen encoded by the delivered DNA vaccine construct.

### Example 2

Macroflux technology has been demonstrated to be suitable for polypeptide vaccine delivery to skin and to induce immune responses similar to or greater than conventional delivery by needle and syringe to muscle. When protein vaccines are delivered extra-cellularily, humoral responses are obtained, as the presentation of the anitgen occurs via the class II MHC/HLA pathway. Only when protein vaccines are delivered into the cytosol (or when the antigen is produced intracellularly—as replicating vaccines or DNA vaccines), a cellular immune response is achieved in addition. In this example we combine transdermal polypeptide vaccine delivery by microprojection array technology, using dry coated arrays or gel reservoirs, with ultrasound to assist intracellular delivery. Immune responses to Hepatitis B virus surface antigen (HBsAg) protein are monitored. Nine treatment groups are evaluated:

Group 1: HBsAg protein-coated microprojection array (MA) delivery (5 min application time) without any augmentation of intracellular delivery.

Group 2: HBsAg protein-coated microprojection array delivery (5 min application time) followed by ultrasound after removal of the microprojection array.

Group 3: HBsAg protein-coated microprojection array delivery (5 min application time) followed by ultrasound with microprojection array remaining in place during ultrasound.

Group 4: Application of uncoated microprojection array followed by ultrasound with HBsAg protein in gel reservoir after removal of the microprojection array. The gel reservoir is in place for 15 min prior to ultrasound.

Group 4A: Application of uncoated microprojection array with HBsAg protein in gel reservoir after removal of the microprojection array, no ultrasound. The gel reservoir is in place for 20 min.

Group 5: Application of uncoated microprojection array followed by ultrasound with HBsAg protein in gel reservoir with microprojection array remaining in place during ultrasound. The gel reservoir is in place for 15 min prior to ultrasound.

Group 5A: Application of uncoated microprojection array with HBsAg protein in gel reservoir with microprojection array remaining in place, no ultrasound. The gel reservoir is in place for 20 min.

Group 6: topical HBsAg protein application followed by ultrasound 15 min after application.

Group 6A: topical HbsAg protein application for 20 min, no ultrasound.

### Materials and Methods

Microprojection arrays: MA 1035 (microprojection length 225 μm, 675 microprojections/cm², 2 cm² array) coated with HBsAg protein (Aldevron, Fargo, N.Dak.).

Microprojection array coating: 30 μg RBsAg protein per array, obtained by roller coater methodology using an aqueous formulation containing 20 mg/mL HBsAg protein, 20 mg/mL sucrose, 2 mg/mL HEC, and 2 mg/mL Tween 20.

HBsAg protein gel: 350 μL of an aqueous formulation containing 1.5% HEC, 20 mg/mL HBsAg protein, and 2 mg/mL Tween 20.

Topical HBsAg protein application: 50 μg HBsAg protein in 50 μl saline.

Ultrasound conditions: 1 MHz; 1 W/cm²; 1 minute, delivered by transducer described in FIG. 1.

HBsAg protein delivery to hairless guinea pig (HGP) skin: Microprojection arrays are applied to live HGP for 5 minutes and the application site is marked. HBsAg protein delivery by microprojection array/HBsAg protein gel is augmented as indicated in the treatment table. Ultrasound is done immediately following HBsAg protein delivery by microprojection array, while all animals remain under anesthesia.

Humoral immune responses two weeks after one booster application at week four are measured using the ABBOTT AUSAB EIA Diagnostic Kit and quantification panel. Antibody titers of higher than the protective level of 10 mIU/ml are marked as “positive” in Table 2.

Cellular responses are determined using a surrogate assay to predict CTL activity: spleen cells are harvested at the time of obtaining the sera for antibody titer determination and the number of gamma interferon producing CD8 cells—after depletion of CD4 positive cells by anti-CD4-coated Dynabeads (Dynal, N.Y.)—are determined by ELISPOT assay after a five day in vitro re-stimulation with the HBsAg protein. A “positive” response is scored when (i) mean number of cells in wells re-stimulated with HBsAg are significantly (P<0.05, student's t test) higher than in wells re-stimulated with ovalbumin (Ova), an irrelevant antigen (ii) net number of spot forming cells (SFCs) (SFCs in wells stimulated with HBsAg minus number of SFCs in wells stimulated with Ova) is 5 or larger and (iii) the ratio of mean number of SFCs in HBsAg wells to mean number of SFCs in Ova wells is greater than 2.0.

TABLE 2
Treatment Table and Immune Responses

| | | HBsAg Protein | Augmentation | Immune response | |
|---|---|---|---|---|---|
| Grp | n | Delivery to Skin | Method | humoral | cellular |
| 1 | 4 | Coated MA | none | positive | negative |
| 2 | 4 | Coated MA, removed | ultrasound | positive | positive |
| 3 | 4 | Coated MA, integrated | ultrasound | positive | positive |
| 4 | 4 | Uncoated MA, | ultrasound | positive | positive |
| | | removed, gel | | | |
| 4A | 4 | Uncoated MA, | none | positive | negative |
| | | removed, gel | | | |
| 5 | 4 | Uncoated MA, | ultrasound | positive | positive |
| | | integrated, gel | | | |
| 5A | 4 | Uncoated MA, | none | positive | negative |
| | | integrated, gel | | | |
| 6 | 4 | Topical protein | ultrasound | negative | negative |
| 6A | 4 | Topical protein | none | negative | negative |

This example demonstrates that ultrasound can augment intracellular polypeptide vaccine uptake after delivery to skin by coated microprojection array or gel reservoir through microprojection array generated passages and can result in the induction of humoral and cellular immune responses to the polypeptide vaccine. From the foregoing description and examples, one of ordinary skill in the art can easily ascertain that the present invention, among other things, provides an effective and efficient means for transdermally delivering a vaccine to a patient.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A delivery system for delivering an immunologically active agent to a subject, comprising:
a microprojection member having a plurality of stratum corneum-piercing microprojections;
a formulation having said immunologically active agent; and
an ultrasonic device adapted to apply ultrasonic energy to said subject.

2. The system of claim 1, wherein said microprojection member has a microprojection density of at least approximately 10 microprojections/cm².

3. The system of claim 2, wherein said microprojection member has a microprojection density in the range of at least approximately 200-2000 microprojections/cm² .

4. The system of claim 1, wherein said microprojections are adapted to pierce through the stratum corneum to a depth of less than about 500 micrometers.

5. The system of claim 1, wherein said formulation comprises a coating disposed on at least one of said microprojections.

6. The system of claim 1, wherein said immunologically active agent comprises a protein-based vaccine.

7. The system of claim 6, wherein said application of said ultrasonic energy to said subject provides in vivo intracellular delivery of said protein-based vaccine, whereby said delivery of said protein-based vaccine into skin-presenting cells leads to cellular loading of said protein-based vaccine onto class I MHC/HLA presentation molecules in addition to class II MHC/HLA presentation molecules.

8. The system of claim 7, wherein a cellular and humoral response is produced in said subject

9. The system of claim 1, wherein said immunologically active agent comprises a DNA vaccine.

10. The system of claim 9, wherein said application of said ultrasonic energy to said subject provides in vivo intracellular delivery of said DNA vaccine, whereby said delivery of said DNA vaccine leads to cellular expression of protein and loading of said protein onto class I MHC/HLA presentation molecules in addition to class II MHC/HLA presentation molecules.

11. The system of claim 10, wherein a cellular and humoral response is produced in said subject

12. The system of claim 10, wherein said immune response produced in said subject is exclusively a cellular response

13. The system of claim 1, wherein said immunologically active agent comprises an agent selected from the group consisting of proteins, polysaccharide conjugates, oligosaccharides, lipoproteins, subunit vaccines, Bordetella pertussis (recombinant DPT vaccine—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-1 1, Quadrivalent recombinant BLP L1 [from HPV-6], HPV-1 1, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM 1970, Treponema pallidum (surface lipoproteins), Varicella zoster virus (subunit, glycoproteins), Vibrio cholerae (conjugate lipopolysaccharide), whole virus, bacteria, weakened or killed viruses, cytomegalo virus, hepatitis B virus, hepatitis C virus, human papillomavirus, rubella virus, varicella zoster, weakened or killed bacteria, bordetella pertussis, clostridium tetani, corynebacterium diptheriae, group A streptococcus, legionella pneumophila, neisseria meningitis, pseudomonas aeruginosa, streptococcus pneumoniae, treponema pallidum, vibrio cholerae, flu vaccines, Lyme disease vaccine, rabies vaccine, measles vaccine, mumps vaccine, chicken pox vaccine, small pox vaccine, hepatitis vaccine, pertussis vaccine, diptheria vaccine, nucleic acids, single-stranded and double-stranded nucleic acids, supercoiled plasmid DNA, linear plasmid DNA, cosmids, bacterial artificial chromosomes (BACs), yeast artificial chromosomes (YACs), mammalian artificial chromosomes, and RNA molecules.

14. The system of claim 13, wherein said formulation includes an immunologically potentiating adjuvant.

15. The system of claim 14, wherein said adjuvant is selected from the group consisting of aluminum phosphate gel, aluminum hydroxide, algal glucan, b-glucan, cholera toxin B subunit, CRL1005, ABA block polymer with mean values of x=8 and y=205, gamma insulin, linear (unbranched) β-D(2->1) polyfructofuranoxyl-a-D-glucose, Gerbu adjuvant, N-acetylglucosamine-(b 1-4)-N-acetylmuramyl-L-alanyl-D-glutamine (GMDP), dimethyl dioctadecylammonium chloride (DDA), zinc L-proline salt complex (Zn-Pro-8), Imiquimod (1-(2-methypropyl)-1 H-imidazo[4,5-c]quinolin-4-amine, ImmTher™, N-acetylglucoaminyl-N-acetylmuramyl-L-Ala-D-isoGlu-L-Ala-glycerol dipalmitate, MTP-PE liposomes, C59H108N6O19PNa—3H2O (MTP), Murametide, Nac-Mur-L-Ala-D-Gln-OCH3, Pleuran, b-glucan, QS-21; S-28463, 4-amino-a, a-dimethyl-1H-imidazo[4,5-c]quinoline-1-ethanol, sclavo peptide, VQGEESNDK.HCl (1L-1b 163-171 peptide), threonyl-MDP (Termurtide™), N-acetyl muramyl-L-threonyl-D-isoglutamine, interleukin 18, IL-2 IL-12, IL-15, DNA oligonucleotides, CpG containing oligonucleotides, gamma interferon, NF kappa B regulatory signaling proteins, heat-shock proteins (HSPs), GTP-GDP, Loxoribine, MPL®, Murapalmitine, and Theramide™.

16. The system of claim 5, wherein said formulation includes a surfactant.

17. The system of claim 16, wherein said surfactant is selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, sorbitan derivatives, sorbitan laureate, alkoxylated alcohols, and laureth-4.

18. The system of claim 5, wherein said formulation includes an amphiphilic polymer.

19. The system of claim 18, wherein said amphiphilic polymer is selected from the group consisting of cellulose derivatives, hydroxyethylcellulose (HEC), hydroxypropyl-methylcellulose (HPMC), hydroxypropycellulose (HPC), methylcellulose (MC), hydroxyethylmethylcellulose (HEMC), ethylhydroxyethylcellulose (EHEC), and pluronics.

20. The system of claim 5, wherein said formulation includes a hydrophilic polymer.

21. The system of claim 20, wherein said hydrophilic polymer is selected from the group consisting of poly(vinyl alcohol), poly(ethylene oxide), poly(2-hydroxyethylmethacrylate), poly(n-vinyl pyrolidone), polyethylene glycol and mixtures thereof.

22. The system of claim 5, wherein said formulation includes a biocompatible carrier.

23. The system of claim 22, wherein said biocompatible polymer is selected from the group consisting of human albumin, bioengineered human albumin, polyglutamic acid, polyaspartic acid, polyhistidine, pentosan polysulfate, polyamino acids, sucrose, trehalose, melezitose, raffinose and stachyose.

24. The system of claim 5, wherein said formulation includes a vasoconstrictor.

25. The system of claim 24, wherein said vasoconstrictor is selected from the group consisting of epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline.

26. The system of claim 5, wherein said formulation includes a pathway patency modulator.

27. The system of claim 26, wherein said pathway patency modulator is selected from the group consisting of osmotic agents, sodium chloride, zwitterionic compounds, amino acids, anti-inflammatory agents, betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate, prednisolone 21-succinate sodium salt, anticoagulants, citric acid, citrate salts, sodium citrate, dextran sulfate sodium, and EDTA.

28. The system of claim 5, wherein said formulation includes an antioxidant.

29. The system of claim 28, wherein said antioxidant is selected from the group consisting of sodium citrate, citric acid, ethylene-dinitrilo-tetraacetic acid (EDTA), ascorbic acid, methionine, and sodium ascorbate.

30. The system of claim 5, wherein said formulation further includes a low volatility counterion.

31. The system of claim 30, wherein said low volatility counterion is selected from the group consisting of maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, aspartic acid, glutamic acid, carbonic acid, sulfuric acid, and phosphoric acid, and mixtures thereof.

32. The system of claim 30, wherein said low volatility counterion is selected from the group consisting of monoethanolomine, diethanolamine, triethanolamine, tromethamine, methylglucamine, glucosamine, histidine, lysine, arginine, sodium hydroxide, potassium hydroxide, calcium hydroxide, magnesium hydroxide, ammonia and morpholine, and mixtures thereof.

33. The system of claim 5, wherein said coating has a viscosity less than approximately 500 centipoise and greater than 3 centipoise.

34. The system of claim 5, wherein said coating has a thickness less than approximately 25 microns.

35. The system of claim 1, wherein said formulation comprises a hydrogel.

36. The system of claim 35, wherein said hydrogel comprises a macromolecular polymeric network.

37. The system of claim 36, wherein said macromolecular polymeric network is selected from the group consisting of hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics.

38. The system of claim 35, wherein said formulation includes a surfactant.

39. The system of claim 38, wherein said surfactant is selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, sorbitan derivatives, sorbitan laureate, alkoxylated alcohols, and laureth-4.

40. The system of claim 35, wherein said formulation includes an amphiphilic polymer.

41. The system of claim 40, wherein said amphiphilic polymer is selected from the group consisting of cellulose derivatives, hydroxyethylcellulose (HEC), hydroxypropyl-methylcellulose (HPMC), hydroxypropycellulose (HPC), methylcellulose (MC), hydroxyethylmethylcellulose (HEMC), ethylhydroxyethylcellulose (EHEC), and pluronics.

42. The system of claim 35, wherein said formulation includes a pathway patency modulator.

43. The system of claim 42, wherein said pathway patency modulator is selected from the group consisting of osmotic agents, sodium chloride, zwitterionic compounds, amino acids, anti-inflammatory agents, betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate, prednisolone 21-succinate sodium salt, anticoagulants, citric acid, citrate salts, sodium citrate, dextran sulfate sodium, and EDTA.

44. The system of claim 35, wherein said formulation includes a vasoconstrictor.

45. The system of claim 44, wherein said vasoconstrictor is selected from the group consisting of epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline.

46. The system of claim 1, wherein said ultrasonic device is adhered to said microprojection member.

47. The system of claim 1, wherein said ultrasonic device further includes a matching layer to facilitate transmission of said ultrasonic energy.

48. The system of claim 47, wherein said ultrasonic device further includes a double-sided adhesive layer.

49. The system of claim 1, wherein said ultrasonic device generates sound waves having a frequency at least about 20 kHz.

50. A method for transdermally delivering an immunologically active agent to a subject, comprising the steps of:
providing a microprojection delivery system, said delivery system including a microprojection member having a plurality of stratum corneum-piercing microprojections, a formulation including the immunologically active agent and an ultrasonic device;
applying said microprojection member to a desired location on said subject; and
transmitting ultrasonic energy from said ultrasonic device to said desired location on said subject to facilitate delivery of said immunologically active agent.

51. The method of claim 50, wherein said immunologically active agent comprises protein-based vaccines.

52. The method of claim 51, wherein said transmission of said ultrasonic energy to said subject provides in vivo intracellular delivery of said protein-based vaccine, whereby said delivery of said protein-based vaccine into skin-presenting cells leads to cellular loading of said protein-based vaccine onto class I MHC/HLA presentation molecules in addition to class II MHC/HLA presentation molecules.

53. The method of claim 52, wherein a cellular and humoral response is produced in said subject.

54. The method of claim 50, wherein said immunologically active agent comprises a DNA vaccine.

55. The method of claim 54, wherein said transmission of said ultrasonic energy to said subject provides in vivo intracellular delivery of said DNA vaccine, whereby said delivery of said DNA vaccine leads to cellular expression of protein and loading of said protein onto class I MHC/HLA presentation molecules in addition to class II MHC/HLA presentation molecules.

56. The method of claim 55, wherein a cellular and humoral response is produced in said subject.

57. The method of claim 55, wherein said immune response produced in said subject is exclusively a cellular response.

58. The method of claim 50, wherein said step of transmitting ultrasonic energy from said ultrasonic device comprises directing said ultrasonic energy through said microprojection member.

59. The method of claim 58, wherein said ultrasonic device is adhered to said microprojection member.

60. The method of claim 58, wherein said formulation comprises a hydrogel incorporated in a gel pack and wherein said ultrasonic device is adhered to said gel pack.

61. The method of claim 50, further comprising the step of removing said microprojection member before transmitting energy with said ultrasonic device.

62. The method of claim 61, wherein said step of transmitting ultrasonic energy with said ultrasonic device includes the step of adhering said ultrasonic device to said desired location on said subject.

63. The method of claim 50, wherein said formulation comprises a coating applied to at least one of said microprojections and wherein said step of transmitting said ultrasonic energy with said ultrasonic device occurs in the range of approximately 5 sec to 30 min after said step of applying said microprojection member to said subject.

64. The method of claim 50, wherein said step of transmitting ultrasonic energy with said ultrasonic device occurs in the range of approximately 30 sec to 15 min after said step of applying said microprojection member to said subject.

65. The method of claim 50, wherein said formulation comprises a hydrogel incorporated in a gel pack and wherein said step of transmitting ultrasonic energy with said ultrasonic device occurs in the range of approximately 5 min to 24 h after said step of applying said microprojection member to said subject.

66. The method of claim 65, wherein said step of transmitting ultrasonic energy with said ultrasonic device occurs in the range of approximately 10 min to 4 h after said step of applying said microprojection member to said subject.

67. The method of claim 50, wherein said formulation comprises a coating applied to at least one of said microprojections and a hydrogel incorporated in a gel pack.

68. The method of claim 67, further including the step of removing said microprojection member from said subject before said step of transmitting said ultrasonic energy to said subject.

69. The method of claim 67, wherein said step of transmitting energy with said ultrasonic device occurs in the range of approximately 5 sec to 24 h after said step of applying said microprojection member to said subject.

70. The method of claim 67, wherein said step of transmitting ultrasonic energy with said ultrasonic device occurs in the range of approximately 30 sec to 4 h after said step of applying said microprojection member to said subject.

71. The method of claim 50, wherein said step of transmitting ultrasonic energy comprises applying sound waves having a frequency in the range of approximately 20 kHz to 10 MHz.

72. The method of claim 67, wherein said step of transmitting ultrasonic energy comprises applying sound waves having a frequency in the range of approximately 20 kHz to 1 MHz.

73. The method of claim 50, wherein said step of transmitting ultrasonic energy comprises applying ultrasonic energy having an intensity in the range of approximately 0.01 W/cm² to 100 W/cm².

74. The method of claim 50, wherein said step of transmitting ultrasonic energy comprises applying ultrasonic energy having an intensity in the range of approximately 1 W/cm² to 20 W/cm².

75. The method of claim 50, wherein said step of transmitting ultrasonic energy comprises applying ultrasonic energy for a duration in the range of approximately 5 sec to 1 h.

76. The method of claim 50, wherein said step of transmitting ultrasonic energy comprises applying energy for a duration in the range of approximately 30 sec to 10 min.
